(19) United States
(12) Patent Application Publication
(10) Pub. No.: US 2018/0014733 A1
(43) Pub. Date: Jan. 18, 2018

(54) SUBJECT INFORMATION ACQUISITION APPARATUS AND METHOD FOR ACQUIRING SUBJECT INFORMATION

(71) Applicant: CANON KABUSHIKI KAISHA, Tokyo (JP)

(72) Inventor: Takeshi Suwa, Tokyo (JP)

(21) Appl. No.: 15/644,522

(22) Filed: Jul. 7, 2017

(30) Foreign Application Priority Data
Jul. 12, 2016 (JP) 2016-137645
Jun. 15, 2017 (JP) 2017-118014

(51) Int. Cl. A61B 5/00

(52) U.S. Cl. CPC A61B 5/0095; A61B 5/704; A61B 5/708; A61B 5/004; A61B 5/4312; A61B 2576/02

(57) ABSTRACT

A subject information acquisition apparatus includes at least one first detection element and at least one second detection element configured to output signals with polarities opposite to each other upon receiving acoustic waves propagated from a subject, and a signal processing unit configured to acquire information on the subject by using a differential signal obtained based on a difference between the signals output from the first and the second detection elements.

### BACKGROUND

### Field of the Disclosure

The present disclosure relates to a subject information acquisition apparatus and a method for acquiring subject information.

### Description of the Related Art

When a subject is irradiated with pulsed light, acoustic waves are generated with the light absorbed in the subject. This is known as a photoacoustic effect. A technique known as photoacoustic tomography (PAT) uses the acoustic waves generated by the photoacoustic effect for visualizing an internal structure serving as the generation source of the acoustic waves. This technique can be used for imaging physiological information, that is, functional information on a living body.

A known PAT apparatus mechanically moves a probe with a driving unit such as a motor and acquires acoustic waves over a wide range of a subject. Such an apparatus has been plagued by noise, generated due to an operation of the driving unit, superimposed as electric noise on an electrical signal output from the probe in some cases.

In view of the above, Japanese Patent Application Laid-Open No. 2011-200381 discusses a technique of reducing the influence of the noise, attributable to the driving unit, on the electrical signal obtained by receiving the acoustic waves. More specifically, the operation of the driving unit is at least partially stopped while the probe is receiving the acoustic waves.

However, the technique discussed in Japanese Patent Application Laid-Open No. 2011-200381 results in the probe repeatedly stopping during its movement, causing a time required for the measurement to be longer.

### SUMMARY

According to an aspect of the present disclosure, a subject information acquisition apparatus includes at least one first detection element and at least one second detection element, wherein the first detection element is configured to output signals with polarities opposite to the second detection element, upon receiving acoustic waves propagated from a subject, and a signal processing unit configured to acquire information on the subject by using a differential signal obtained based on a difference between the signals output from the first and the second detection elements.

Further features of the present disclosure will become apparent from the following description of exemplary embodiments with reference to the attached drawings.

### BRIEF DESCRIPTION OF THE DRAWINGS

FIG. 1 is a diagram illustrating an example of a configuration according to a first exemplary embodiment of the present disclosure.

FIG. 2 is an equivalent circuit diagram illustrating an example of how groups of acoustic wave detection elements according to the first exemplary embodiment are electrically connected.

FIGS. 3A, 3B, and 3C are diagrams illustrating examples of electrical signals obtained in the first exemplary embodiment.

FIG. 4 is a flowchart illustrating a measurement sequence according to the first exemplary embodiment.

FIGS. 5A and 5B are diagrams illustrating examples of arrangement of acoustic wave detection elements.

FIG. 6 is an equivalent circuit diagram illustrating an example of how groups of acoustic wave detection elements according to a second exemplary embodiment are electrically connected.

FIG. 7 is an equivalent circuit diagram illustrating an example of how groups of acoustic wave detection elements according to a fourth exemplary embodiment are electrically connected.

FIGS. 8A, 8B, and 8C are diagrams respectively illustrating examples of electric signals obtained in the fourth exemplary embodiment.

FIGS. 9A, 9B, and 9C are diagrams respectively illustrating alternative arrangements of acoustic wave detection elements according to the fourth exemplary embodiment.

FIGS. 10A, 10B, 10C, and 10D are diagrams respectively illustrating alternative arrangements of acoustic wave detection elements according to the fourth exemplary embodiment.

### DESCRIPTION OF THE EMBODIMENTS

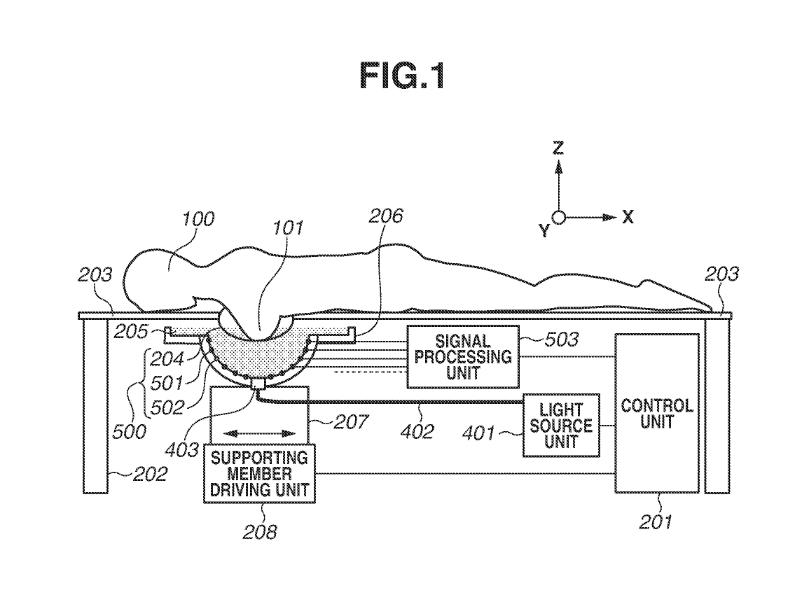

A configuration of a subject information acquisition apparatus according to a first exemplary embodiment will be described with reference to FIG. 1. The subject information acquisition apparatus according to the present exemplary embodiment is a photoacoustic tomography (PAT) apparatus configured to irradiate a subject 101, which is a body part of an examinee 100 in a prone position, with pulsed light and receive acoustic waves in return. The subject 101 illustrated in FIG. 1 is a breast. In FIG. 1, an X axis, a Y axis, and a Z axis are defined as illustrated. The X axis and the Y axis represent horizontal directions, whereas the Z axis represents a height direction.

The subject information acquisition apparatus according to the present exemplary embodiment includes a control unit 201, a bed 203, a holding member 204, a supporting member 206, a support base 207, a supporting member driving unit 208, a light source unit 401, and a signal processing unit 503.

The control unit 201 controls the supporting member driving unit 208, the light source unit 401, and the signal processing unit 503. The control unit 201 may be a general purpose personal computer (PC), or may be implemented with dedicated hardware or software.

The bed 203 can support the examinee 100 in the prone position. The bed 203 has an opening where the subject 101 is inserted. Leg portions 202 supporting the bed 203 may be provided with a height adjustment mechanism.

The holding member 204 holds the subject 101 that has been inserted in the opening provided in the bed 203. With the holding member 204 holding the subject 101, measurement can be performed on the subject 101 while the shape thereof is held in a stable state. The holding member 204 may be exchangeable or adjustable in accordance with a size and a shape of the subject 101, for better usability of the apparatus. The holding member 204 is preferably thin and made of a material with acoustic impedance close to that of the subject 101, to reduce reflections at an interface between the subject 101 and the holding member 204. Furthermore, the holding member 204 is preferably made of a material with high rigidity and with a high transmittance (preferably 90% or higher) because the light from the PAT apparatus is emitted onto the subject 101 through the holding member 204. Examples of the preferable material include polymethylpentene and polyethylene terephthalate.

Acoustic matching liquid 205 is used for facilitating propagation of the acoustic waves from the subject 101 to an acoustic wave detection unit 500. The acoustic matching liquid 205 is preferably selected from materials that have acoustic impedance close to that of the human body and that do not largely attenuate the acoustic waves. Examples of such materials include water and oil. With the appropriate acoustic matching liquid 205, the light emitted from a light emitting unit 403 can be efficiently guided to the subject 101, and the acoustic waves generated from the subject 101 can be efficiently propagated to the acoustic wave detection unit 500.

The supporting member 206 supports the acoustic wave detection unit 500 and can hold the acoustic matching liquid 205. In the present exemplary embodiment, the supporting member 206 includes a semispherical portion provided with a plurality of the acoustic wave detection units 500 and filled with the acoustic matching liquid 205. More specifically, a space between the holding member 204 and the supporting member 206 is filled with the acoustic matching liquid 205.

The support base 207 that supports the supporting member 206 can move integrally with the supporting member 206, and can be configured to move along two rails extending along directions that cross each other, for example. In the description below, the support base 207 is movable in an X-Y plane. Alternatively, the support base 207 may be movable three-dimensionally. The supporting member driving unit 208 moves the supporting member 206 in a horizontal direction. In the present exemplary embodiment, the supporting member 206 and the support base 207 can be moved integrally. The supporting member driving unit 208 includes an actuator such as a motor.

The light source unit 401 is a light source that emits pulsed light and may be implemented with a Ti:Sa laser, a yttrium aluminum, garnet (YAG) laser, an alexandrite laser, a dye laser, a light emitting diode (LED) or the like. A wavelength of the pulsed light emitted from the light source unit 401 is set in such a manner that the light can propagate into the subject 101. More specifically, when the subject 101 is a living body, the light has a wavelength that is in a range between 600 nm inclusive and 1100 nm inclusive, so as not to be actively absorbed by hemoglobin and water. When the subject 101 is a living body, the pulsed light has a pulse width of about 10 to 50 nanoseconds. When the light source unit 401 is a laser, a maximum value of an irradiation density (an amount of light emitted per unit area) of the pulsed light emitted onto the living body needs to be set so as not to exceed the maximum permissible exposure (MPE) defined by laser safety standards (JIS standard C6802 and International Electrotechnical Commission (IEC) 60825-1).

A light transmission unit 402 is a member through which the pulsed light, emitted from the light source unit 401, is transmitted and which includes a fiber bundle and a plurality mirrors.

The light emitting unit 403 emits the pulsed light, transmitted thereto through the light transmission unit 402, onto the subject 101 via a diffusion plate and a lens. The light emitting unit 403 is held by the supporting member 206, and thus moves when the supporting member 206 moves.

The acoustic wave detection unit 500, supported by the supporting member 206, receives the acoustic waves propagated thereto from the subject 101, and generates an electrical signal. In the present exemplary embodiment, the acoustic wave detection unit 500 includes a pair of detection elements. More specifically, an acoustic wave detection element 501 as a first detection element and an acoustic wave detection element 502 as a second detection element are provided. The acoustic wave detection element 502, which is illustrated as a single element in FIG. 1, is in one-to-one relationship with the acoustic wave detection element 501. The acoustic wave detection unit 500 receives and converts the acoustic waves propagated thereto from the subject 101 via the acoustic matching liquid 205, into the electrical signal. The acoustic wave detection elements 501 and 502, forming the acoustic, wave detection unit 500, each preferably have a high sensitivity and a wide frequency band, and may be a capacitive pressure sensitive element such as capacitive micromachined ultrasound transducer (CMUT) or a piezoelectric element. A plurality of the acoustic wave detection units 500 is arranged on a surface of the supporting member 206 facing the acoustic matching liquid 205 in such a manner that their highest sensitivity directions, in terms of reception directionality, are concentrated. With the acoustic wave detection units 500 thus arranged, information on an area where the highest sensitivity directions of the acoustic wave detection element 501, in terms of reception directionality, are concentrated can be acquired with high resolution. When highest sensitivity directions, in terms of reception directionality, or directional axes of the plurality of acoustic wave detection units 500 intersect at a single point, the information can be acquired with the highest resolution at the intersecting point. In the present exemplary embodiment, the highest sensitivity directions, in terms of reception directionality, of the plurality of acoustic wave detection units 500 intersect at a single point. An area around the intersecting point where the information on the subject 101 can be obtained with a predetermined resolution or higher is referred to as a high-resolution area. For example, the predetermined resolution is 50% of the highest resolution.

In a case where the acoustic wave detection units 500 are fixed to the supporting member 206, a spatial position of the high-resolution area relative to the supporting member 206 is fixed. In the present exemplary embodiment, the supporting member 206 is moved with respect to the subject 101 by the support base 207 and the supporting member driving unit 208, whereby high resolution information can be obtained over a wide area.

Upon receiving the acoustic waves, the acoustic wave detection element 502 as the second detection element outputs an electrical signal with a polarity that is opposite to that of the electrical signal output from the acoustic wave detection element 501 as the first detection element. The opposite polarities are not limited to positive and negative potentials with 0 V as a reference, but also include amplitudes in opposite directions with respect to any reference potential. For example, electrical signals with opposite polarities are deemed to be output, when the acoustic wave detection element 501 outputs a 5 V electrical signal and the acoustic wave detection element 502 outputs a 1 V electrical signal, with 3 V as the reference. Configurations of the acoustic wave detection elements 501 and 502 will be described in detail below.

The signal processing unit 503 collects the electrical signals from the acoustic wave detection element 501 and the acoustic wave detection element 502 and performs various types of calculation processing, under an instruction from the control unit 201. The signal processing unit 503 may have a function of amplifying the analog electrical signals, output from the acoustic wave detection elements 501 and 502, to a predetermined level, and then converting the resultant signals into digital signals. The signal processing unit 503 may perform image reconstruction based on the digital signals thus obtained by the conversion. More specifically, the image reconstruction may be performed with universal back projection (UBP). The signal processing unit 503 may be a general purpose PC, or may be implemented with dedicated hardware or software.

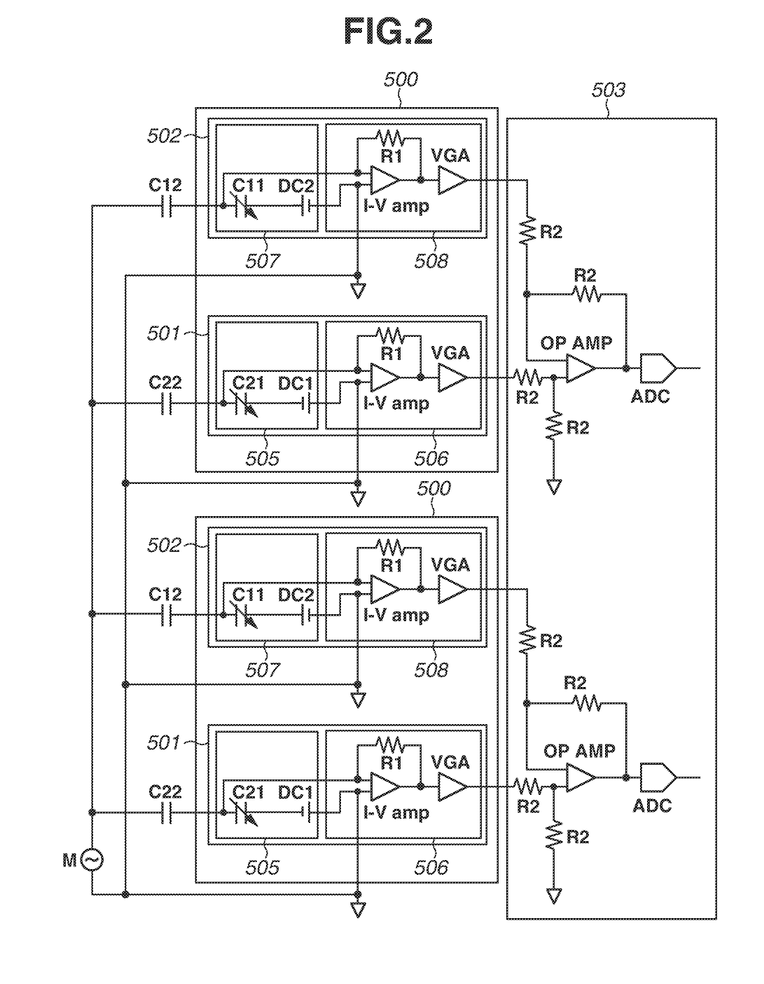

Next, how the acoustic wave detection element 501 and the acoustic wave detection element 502 according to the present exemplary embodiment are electrically connected will be described with reference to FIG. 2. Here, two pairs of the acoustic wave detection element 501 and the acoustic wave detection element 502, as a part of the plurality of acoustic wave detection units 500, are illustrated. In the figure, noise attributable to the motor in the supporting member driving unit 208 is illustrated as a noise source M, and coupling capacitances C12 and C22 between the noise source M and the acoustic wave detection elements 501 and 502 are illustrated. The coupling capacitances C12 and C22 are parasitic capacitances.

In FIG. 2, the acoustic wave detection elements 501 and 502 each include a detection unit that detects the acoustic waves and an amplifying unit that amplifies a signal output from the detection unit. In this description, the detection unit includes a CMUT element that detects a change in electrostatic capacitance occurring due to the reception of the acoustic waves as a change in an amount of current. A capacitor C21 in the acoustic wave detection unit 500 is for detecting the acoustic waves. The acoustic wave detection element 501 according to the present exemplary embodiment includes a detection unit 505 and an amplifying unit 506. The capacitor C21, forming the CMUT element in the detection unit 505, has one terminal connected to a first input terminal of a current-voltage converter (hereinafter, denoted with I-V_amp) of the amplifying unit 506. In FIG. 1, the first terminal is on a side facing the subject 101. The capacitor C21 has a second terminal connected to a second input terminal of the current-voltage converter I-V_amp and a ground GND via a bias voltage source DC1. The amplifying unit 506 includes the current-voltage converter I-V_amp, a feedback resistor R1, and a variable gain amplifier VGA. The current-voltage converter I-V_amp has the first input terminal connected to an output terminal of the current-voltage converter I-V_amp and an input terminal of the variable gain amplifier VGA via the feedback resistor R1. The capacitors C11 and C21 each need not to be a single capacitor element, and may be formed with a plurality of capacitor elements connected in parallel. This means that a plurality of CMUTs are commonly connected, resulting in a smaller area of a diaphragm of each CMUT compared with a case where only a single CMUT is provided in the same area, and thus a higher sensitivity can be achieved against acoustic waves with a higher frequency.

In the configuration described above, a distance between electrodes of the capacitor C21 changes in accordance with sound pressure of the received acoustic waves. Thus, an amount of charges held by the capacitor C21 changes.

The bias voltage source DC1 is a direct current (DC) power source for applying bias voltage to the CMUT element in the acoustic wave detection unit 500.

The amplifying unit 506 amplifies the electrical signal obtained by the acoustic wave detection unit 500, and in this example, includes the current-voltage converter I-V_amp that coverts current, which is a change in the amount of charges resulting from the change in the capacity of the capacitor C21, into voltage, and the variable gain amplifier VGA.

The acoustic wave detection element 502 includes the detection unit 507 and the amplifying unit 508 that amplifies the detection signal output from the detection unit 507, as in the case of the acoustic wave detection element 501. In the present exemplary embodiment, the acoustic wave detection element 502 and the acoustic, wave detection element 501 are only different from each other in that the bias voltage source DC2 supplies voltage, to the capacitor C11 in the detection unit 507, with a polarity opposite to that of the bias voltage source DC1 with respect to the ground GND. With this configuration, the acoustic wave detection elements 501 and 502 output electrical signals with polarities opposite to each other upon receiving the acoustic waves.

The noise source M is illustrated as a voltage source for the sake of description. The noise source M, which is the motor in the supporting member driving unit 208 in the above description, can be a linear scale provided for detecting the position of the support base 207, or may be a peripheral circuit such as a switching power source. Noise can be superimposed on the electrical signal in various ways. For example, the noise generated when the motor is operated may be propagated through a capacitive coupling formed between metal casings forming the bed 203, the support base 207, and the like. The variable gain amplifiers VGA in the acoustic wave detection elements 501 and 502 are preferably set to have the same gain for the sake of processing in a later stage.

In FIG. 2, the signal processing unit 503 includes an operational amplifier OP_AMP as a differential output unit that outputs a differential signal obtained based on a difference between the electrical signals output from one pair of acoustic wave detection elements 501 and 502 and an AD converter ADC that converts an output from the operational amplifier OP_AMP into a digital signal. In this example, the operational amplifier OP_AMP has a gain determined by resistors R2 all having the same capacity value. Alternatively, the operational amplifier OP_AMP can be designed in any way, in terms of its gain.

The signal processing unit 503 includes a reconstruction processing unit (not illustrated) that uses the digital signal obtained by the AD converter ADC to reconstruct an internal image of the subject 101.

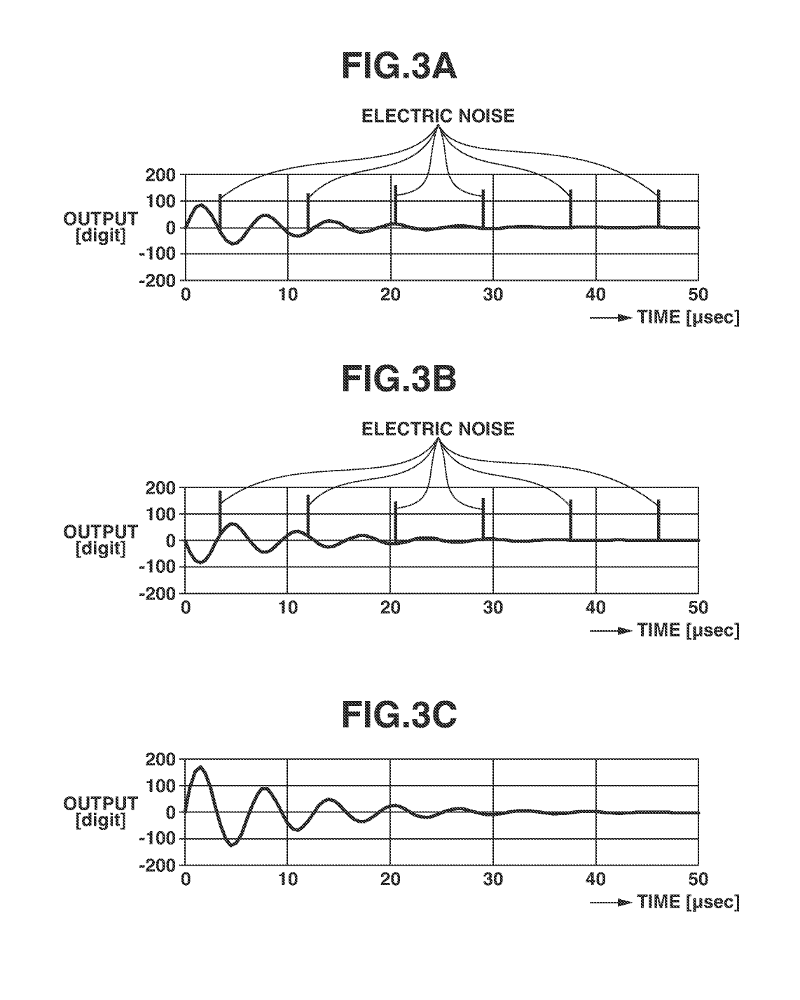

FIGS. 3A, 3B, and 3C illustrate examples of output waveforms of the acoustic wave detection elements 501 and 502 and a differential signal obtained with the elements. In the figure, a horizontal axis represents time and a vertical axis represents a signal output. The figure illustrates a case where the acoustic wave detection elements 501 and 502 have received the same acoustic waves. FIG. 3A illustrates an output waveform of the acoustic wave detection element 501. FIG. 3B illustrates an output waveform of the acoustic wave detection element 502. FIG. 3C illustrates an output waveform of the operational amplifier OP_AMP. As can be seen in FIGS. 3A and 3B, signal components based on the acoustic waves are opposite to each other in the polarity with reference to 0, whereas electric noise components from the noise source M are the same with each other in the polarity. Thus, through processing of taking the difference therebetween, the electric noise components are canceled out and only the acoustic wave components remain. In addition, because the signal components based on the acoustic waves of the electrical signals output from, the acoustic wave detection elements are opposite to each other in the polarity, the differential signal has an amplitude doubled from that of the original signals, whereby a signal with an improved S/N ratio can be achieved. In reality, the electric noise components might not be able to be completely canceled out due to a difference in characteristics between elements forming the acoustic wave detection elements. Still, the components based on the electric noise can be reduced, and the differential signal with a larger amplitude than the original electrical signal can be obtained, whereby a higher S/N ratio can be achieved.

A condition C12=C22 can be satisfied by setting a physical distance between the acoustic wave detection element 501 and the acoustic wave detection element 502 sufficiently shorter than a distance between the noise source (for example, the motor in the supporting member driving unit 208) and the acoustic wave detection element 501 and a distance between the noise source and the acoustic wave detection element 502. As a result, approximately the same voltage caused by the electric noise from the noise source M can be output from the acoustic wave detection element 501 and the acoustic wave detection element 502.

In the present exemplary embodiment, the acoustic wave detection element 501 and the acoustic wave detection element 502 are in one to one relationship. With this configuration, the acoustic wave detection elements 501 and 502 can be arranged close to each other. Thus, paths between the acoustic wave detection elements and the noise source can be set to be equal to each other and can be easily handled. When an electrical signal output from one acoustic wave detection element 502 is commonly used for electrical signals output from a plurality of the acoustic wave detection elements 501, an amount of delay and an amplitude according to a distance between the acoustic wave detection element 502 and each of the acoustic wave detection elements 501 are preferably provided. In such a configuration where one acoustic wave detection element 502 is provided for the plurality of acoustic wave detection elements 501, the number of acoustic wave detection elements can be reduced compared with the configuration employing the one to one relationship. Furthermore, in the configuration where a plurality of acoustic wave detection elements 502 are provided for one acoustic wave detection element 501, electrical signals output from the plurality of acoustic wave detection elements 502 may be combined and a difference between the resultant composite signal and the electrical signal from the acoustic wave detection element 501 may be taken. Thus, random noise components can be reduced.

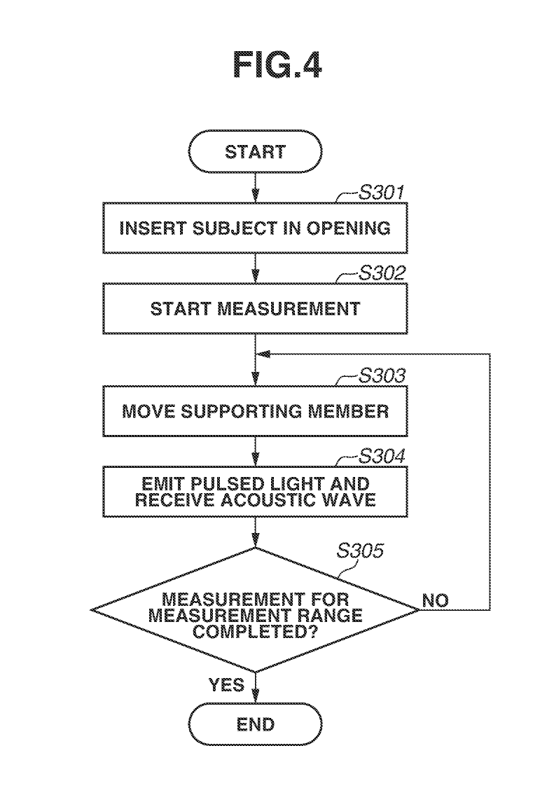

Next, a measurement sequence according to the present exemplary embodiment will be described with reference to FIG. 4.

In step S301, the subject 101 is inserted in the opening of the bed 203.

In step S302, the subject information acquisition apparatus starts measurement processing upon receiving a measurement start instruction from an operator. In this step, the operator can set a measurement range, an accuracy of an acquired signal, or the like. The subject information acquisition apparatus may have a default setting to be executed in a case where no setting is performed by the operator.

In step S303, the control unit 201 moves the supporting member 206 based on the setting made in step S302.

In step S304, the light emitting unit 403 emits the pulsed light onto the subject 101 at a position located as a result of the movement in step S303, and the acoustic wave detection unit 500 receives the acoustic waves.

In step S305, the control unit 201 determines whether the measurement has been completed for the measurement range set in step S302. When the measurement for the measurement range is determined to have been completed (YES in step S305), the measurement sequence is terminated. On the other hand, when the measurement is determined to have not been completed yet (NO in step S305), the processing returns to step S303, and the supporting member 206 is moved to the next measurement point. The supporting member 206 preferably moves along a trajectory with a smooth curve forming a spiral form, a circular form, an elliptical form, or the like.

When the result of the determination in step S305 is YES, the subject 101 is released from the holding member 204.

As described above, the present exemplary embodiment can obtain an electrical signal, output from the acoustic wave detection element, with smaller electric noise superimposed thereon. Thus, in a case where the acoustic wave detection unit is moved with respect to the subject, electric noise generated from the motor or the like can be reduced. Accordingly, the acoustic wave detection unit needs not to be repeatedly stopped during the movement unlike in the technique discussed in Japanese Patent Application Laid-Open No. 2011-200381. Furthermore, when performing the image reconstruction by using the differential signal obtained based on a difference between the electrical signals output in response to the input acoustic waves, with polarities opposite to each other, a reconstructed image with an excellent image quality can be obtained. Thus, the present exemplary embodiment can achieve both shorter measurement time and a higher image quality.

Instead of the subtraction processing taking place in the analog circuit described above with reference to FIG. 2, subtraction processing may be executed after the outputs from the acoustic wave detection element 501 and the acoustic wave detection element 502 are each converted into a digital signal.

### <Arrangement of Acoustic Wave Detection Elements>

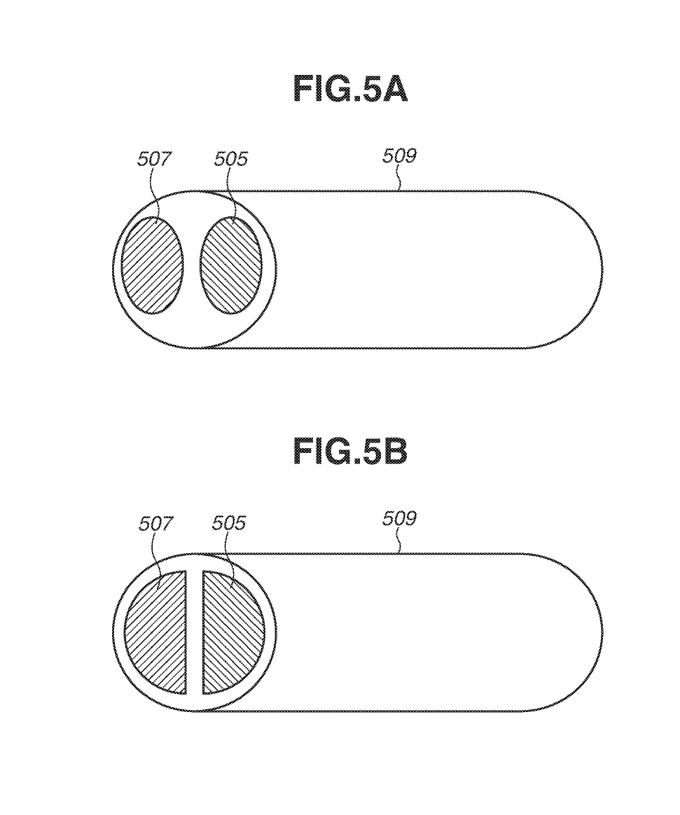

Next, an arrangement of acoustic wave detection elements of the present exemplary embodiment will be described. In the present application example, the pair of acoustic wave detection elements 501 and 502 are provided in a package to form a single module. As illustrated in FIG. 5, the detection units 505 and 507 are provided on one end of a module 509 having a cylindrical shape. The module 509 accommodates the bias voltage source and the amplifying unit. With this configuration, the two acoustic wave detection elements 501 and 502 can be disposed close to each other, whereby a difference in the acoustic waves and the electric noise between the acoustic wave detection elements can be kept small. In other words, the acoustic wave detection elements can output electrical signals close to each other in terms of phase and magnitude of the noise superimposed thereon. Thus, more accurate electric noise subtraction can be achieved to obtain an excellent reconstructed image.

The capacitors C11 and C21 may each have a circular or elliptical wave receiving surface as illustrated in FIG. 5A or may have a semicircular wave receiving surface as illustrated in FIG. 5B.

In the configuration illustrated in FIG. 5A, the capacitor C11 and the capacitor C21 each have a near-circular shape, and thus can achieve receiving sensitivity characteristics involving lower angle dependence. For example, the configuration illustrated in FIG. 5A is suitable for a situation where signals are to be uniformly acquired over a wide area on a surface layer. The configuration illustrated in FIG. 5B features a larger capacitor area on the reception surface of the module 509, and thus can achieve a higher receiving sensitivity compared with the configuration illustrated in FIG. 5A. Thus, a high S/N ratio of the electrical signal can be achieved, whereby an excellent reconstructed image can be obtained. The configuration illustrated in FIG. 5B is suitable for a situation where the signal is to be received from an area deep inside a living body.

The shape of the wave receiving surface of the acoustic wave detection element is not limited to those described above, and may be designed in accordance with a scanned pattern or an examination target.

In the application example described above, the acoustic wave detection element 501 and the acoustic wave detection element 502 are contained in a package to be a single module. Thus, the detection unit 505 and the detection unit 507 can be arranged close to each other. With this arrangement, the superimposed electric noise can be made substantially the same between the electrical signals output from the acoustic wave detection element 501 and the acoustic wave detection element 502. As a result, the electric noise superimposed on these output signals can be favorably reduced, whereby an excellent reconstructed image can be obtained. With the detection units 505 and 507 arranged close to each other, the components based on the acoustic waves can be made substantially the same therebetween, whereby a reconstructed image with a higher resolution can be achieved.

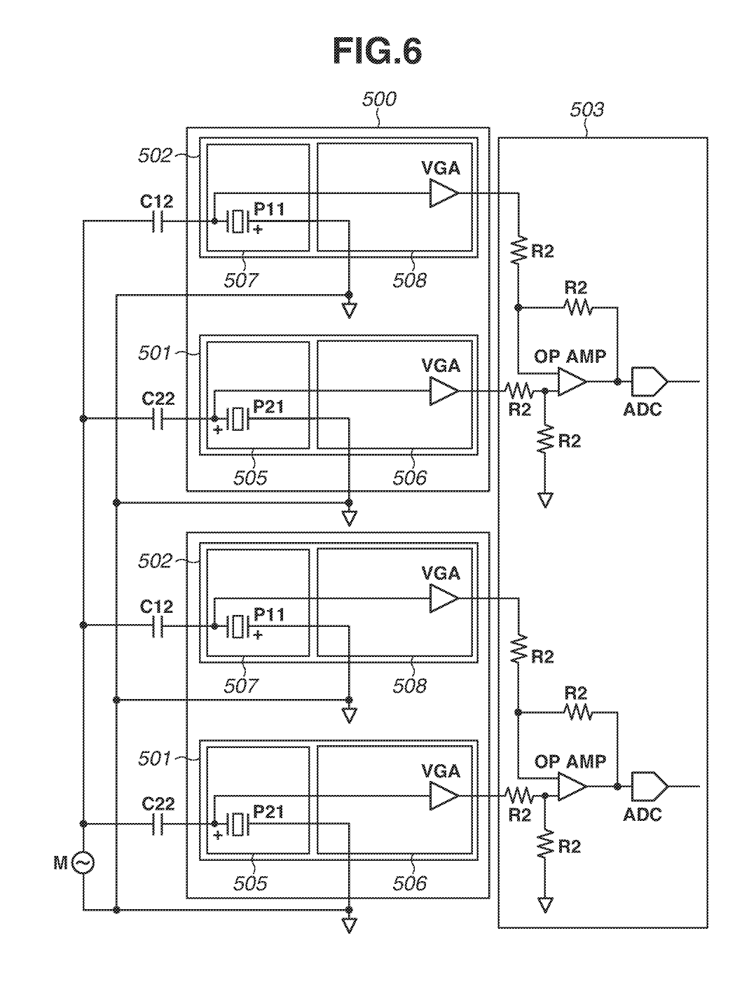

Next a second exemplary embodiment of the present disclosure will be described. FIG. 6 is a partial equivalent circuit diagram of the acoustic wave detection elements 501 and 502 and the signal processing unit 503 according to the present exemplary embodiment. A description on elements that are the same as those in FIG. 2 will be omitted, and a difference will be mainly described.

In the present exemplary embodiment, piezoelectric elements P11 and P21 are used as the detection units. Thus, the current-voltage converter I-V_amp is omitted from the amplifying units 506 and 508.

In the acoustic wave detection element 501, the piezoelectric element P21 has one terminal connected to the variable gain amplifier VGA and the other terminal connected to the ground GND. The terminal connected to the variable gain amplifier VGA is assumed as the wave receiving surface for receiving the acoustic waves. The electric noise M is input to the terminal on the side of the wave receiving surface via the capacitance C22.

The piezoelectric element P11 in the acoustic wave detection element 502 is designed to have an output with a polarity opposite to that of the piezoelectric element P21. More specifically, the piezoelectric elements P21 and P11 are opposite to each other in the surface for receiving the acoustic waves, and opposite electrodes thereof are connected to the variable gain amplifier and the ground GND, respectively. As a result, electromotive force with opposite polarities can be generated with the piezoelectric elements P21 and P11. It is a matter of course that only the opposite connection of the electrodes may be employed.

When output voltage of the piezoelectric element used as the detection unit is insufficient, the output may be amplified with an amplifier provided between the piezoelectric element and the variable gain amplifier VGA.

The configuration using the piezoelectric element according to the present exemplary embodiment can achieve the same effect as that in the first exemplary embodiment.

The acoustic wave detection elements 501 and 502 in the acoustic wave detection unit 500 are designed to have the same characteristics. Unfortunately, in reality, the characteristics of the elements might not completely match due to variations in manufacturing, for example. Thus, in a third exemplary embodiment, a configuration achieving the same effect even when there is a difference in the characteristics between the acoustic wave detection elements 501 and 502 will be described.

First of all, how a difference in characteristics between the acoustic wave detection elements 501 and 502 in the acoustic wave detection unit 500 can be determined will be described.

The determination can be made by using a phantom for evaluating the apparatus as the subject. The phantom is formed with a target provided at a known position in a base material. Thus, the subject information acquisition apparatus can be evaluated by comparing an electrical signal and a reconstructed image, estimated to be obtained with the phantom being the subject, with an electrical signal and a reconstructed image actually obtained. The phantom preferably has optical and acoustic transmission characteristics close to those of the subject, and may be formed with the base material including polyol and filler that can be dispersed in polyol. The target may include pigment such as carbon black as a light-absorbing filler.

As a result of taking a measurement using the phantom as the subject, if a difference in the output between the pair of acoustic wave detection elements 501 and 502 exceeds a predetermined threshold, a signal based on the acoustic wave detection unit 500 including the pair of aforementioned elements may be corrected or may be excluded from being used for the reconstruction in the next measurement and after. Alternatively, a plurality of the thresholds may be set so that a configuration can be obtained in which the signal is corrected when the difference in the output between the pair of acoustic wave detection elements 501 and 502 exceeds a first threshold but falls below a second threshold higher than the first threshold, and is not used for the reconstruction when the difference exceeds the second threshold. The determination may be made by a determination unit provided separately from the signal processing unit 503. Alternatively, the signal processing unit 503 may have the function of the determination unit. The difference in the output between the pair of acoustic wave detection elements may be detected through a method other than the evaluation using the phantom.

Processing executed when the difference in the output between the two acoustic wave detection elements in the acoustic wave detection unit is determined to exceed the first threshold but to fall below the second threshold will now be described. When the two acoustic wave detection elements have similar characteristics, the signals output from the acoustic wave detection unit 500 can be regarded as signals output from a single acoustic wave detection element having a centroid between the two acoustic wave detection elements. When there is a non-negligible difference in the output between the two acoustic wave detection elements, the virtual centroid is shifted toward the acoustic wave detection element with a larger signal output. Thus, the signal processing unit 503 preferably corrects the shifting of the centroid when executing the reconstruction processing. Furthermore, gain correction may be performed to set an output level to be the same as that of other acoustic wave detection units 500.

Next, processing executed when the difference in an output between the two acoustic wave detection elements is determined to exceed the second threshold will be described. Such a determination result indicates that one of the pair of acoustic wave detection elements might have failed. Thus, the signal processing unit 503 executes the reconstruction processing without using the signal from the acoustic wave detection unit 500 including such a pair of elements. A signal output from an acoustic wave detection unit 500 positioned close to the acoustic wave detection unit 500, the output signal of which had been determined not to be used in the reconstruction processing, may be used for interpolation. More specifically, averaging or distance-based weighted averaging processing may be executed on signals from a plurality of the acoustic wave detection units 500 symmetrically arranged with respect to the acoustic wave detection unit 500 the output signal of which had been determined not to be used in the reconstruction processing.

In the example described above, variations between the pair of acoustic wave detection elements is determined based on the difference in the output between the pair of acoustic wave detection elements. For example, when the configuration illustrated in FIG. 2 can directly detect the input from the operational amplifier OP_AMP, it is possible to detect which one of the two acoustic wave detection elements 501 and 502 has failed. In such a case, the signal processing unit 503 executes the reconstruction processing with signals from acoustic wave detection elements other than the acoustic wave detection element determined to have failed by the determination unit. The output from a valid acoustic wave detection element may be doubled to achieve an output level close to that of a signal output from another acoustic wave detection unit 500.

In the present exemplary embodiment, the difference in characteristics between the pair of acoustic wave detection elements is detected so that the subject information can be accurately obtained even when there is a non-negligible difference in the output between the two acoustic wave detection elements. The acoustic wave detection unit 500 described above had been illustrated to include two acoustic wave detection elements. Alternatively, three or more acoustic wave detection elements may be included.

In the exemplary embodiments described above, the noise attributable to the motor in the supporting member driving unit 208 is superimposed on a signal component based on the acoustic wave. It is to be noted that the configuration described above can reduce any noise that is generated from a noise source other than the motor in the supporting member driving unit 208 and is input to the acoustic wave detection elements in the same phase. Furthermore, because the components based on the acoustic waves, output from the acoustic wave detection elements, have opposite polarities, a differential signal with a larger amplitude can be obtained from the elements. Thus, an excellent reconstructed image can be obtained.

The exemplary embodiments described above are all merely exemplary, and thus the present disclosure is not limited to these exemplary embodiments. The specific elements and configurations described above can be changed in various ways without departing from the technical idea of the present disclosure. For example, an element may be replaced with a different element and an additional configuration may be provided. For example, instead of the photoacoustic apparatus described above as an example, the present disclosure may be applied to an ultrasonic echo measurement apparatus that transmits ultrasonic waves from the acoustic wave detection element and receives the resultant reflected waves.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from, the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

With the exemplary embodiment described above, less noise will be superimposed on an electrical signal generated based on acoustic waves even when a probe is continuously moved.

A configuration described in a fourth exemplary embodiment includes a plurality of acoustic wave detection elements with the same polarity and a single acoustic wave detection element with the opposite polarity. More specifically, the described configuration takes a difference between a composite signal obtained by combining signals with the same polarity output from the plurality of acoustic wave detection elements and a signal with the opposite polarity output from the acoustic wave detection element, with respect to the received acoustic waves. Components that are the same as those in the exemplary embodiments described above are denoted with the same reference numerals.

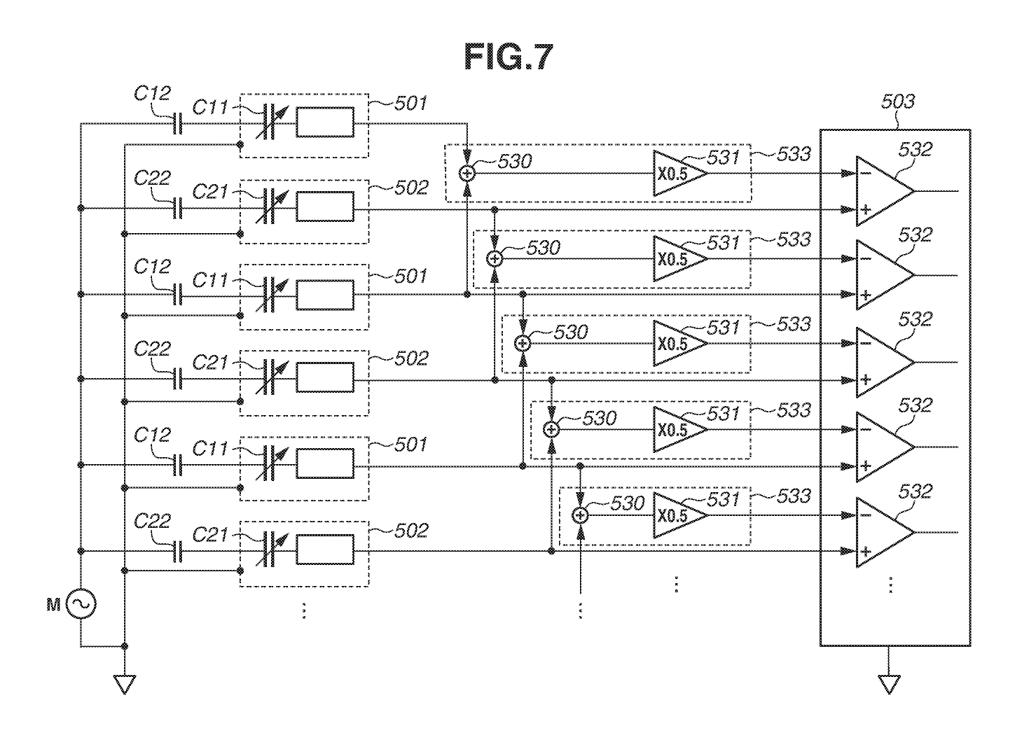

FIG. 7 is an equivalent circuit diagram illustrating an example of how groups of acoustic wave detection elements according to the present exemplary embodiment are electrically connected.

The groups of acoustic wave detection elements illustrated in FIG. 7 each includes the first and second acoustic wave detection elements 501 and 502 that output electric signals with opposite polarities upon receiving acoustic waves propagated from a subject. The groups of acoustic wave detection elements each further include a first combining unit 533 that combines the output signals from two first acoustic wave detection elements 501 or the output signals from two second acoustic wave detection elements 502.

The first combining unit 533 combines the signals from the plurality of related first acoustic wave detection elements 501 or the plurality of related second acoustic wave detection elements 502, to output a first composite signal.

The first combining unit 533 incorporates an adder 530. The adder 530 having a function of adding input signals can be configured by a known adder circuit.

An amplifier 531 is a circuit that amplifies input signals. Typically, when n number of signals are input to the combining unit 533, the amplifier 531 multiplies the amplitudes of the input signals by 1/n and thus averages the amplitudes.

A Subtractor 532, which is a second combining unit included the signal processing unit 503, executes processing of taking a difference between the first composite signal output from the first combining unit 533 and a signal from a single acoustic wave detection element 501 or 502.

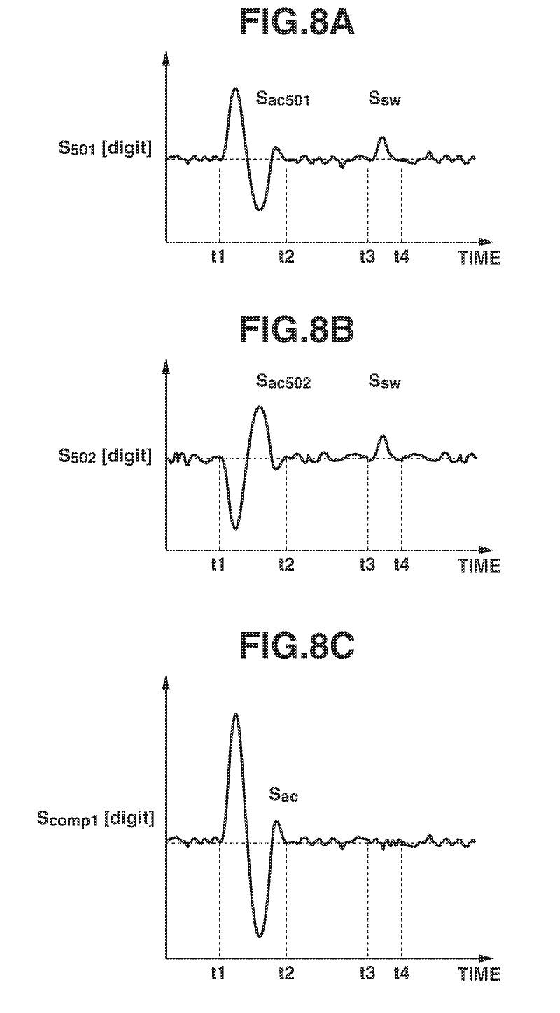

FIG. 8A illustrates a waveform of the output signal from the acoustic wave detection element 501. FIG. 8B is a diagram illustrating a waveform of the output signal from the acoustic wave detection element 502. Signal components S_{ac501} and S_{ac502}, based on received acoustic waves, with opposite polarities are output. For example, the polarities of a noise component S_{sw}, originating from a motor in the supporting member driving unit 208 included in the outputs from the acoustic wave detection elements 501 and 502 are the same.

The output signals from the acoustic wave detection elements 501 and 502 further include a random noise component (S_{rand}) such as thermal noise and shot noise.

Thus, an output signal S₅₀₁ from the acoustic wave detection element 501 can be regarded as a composite of the components, and thus can be expressed as S₅₀₁=S_{ac501}+S_{sw501}+S_{rand501}. Similarly, an output signal S₅₀₂ from the acoustic wave detection element 502 can be expressed as S₅₀₂=S_{ac502}+S_{sw502}+S_{rand502}.

The acoustic wave detection elements 501 and 502 are assumed to have the same level of sensitivity against acoustic waves. The signal components based on the acoustic waves are opposite to each other in the polarity, and thus a relationship S_{ac501}=−S_{ac502} holds true between the first terms in the formulae representing the output signals S₅₀₁ and S₅₀₂. Furthermore, a relationship S_{sw501}=S_{sw502} holds true between the second terms in the formulae because the noise components originating from such a noise source as a motor are the same in the polarity. Finally, an orthogonal relation holds true between the third sections in formulae representing the random noise components S_{rand501} and S_{rand502} with the same amplitude.

Thus, the processing of taking a difference between the output signal from the acoustic wave detection element 501 and the acoustic wave detection element 502 results in a composite signal S_{comp1} that can be represented by S_{comp1}=S₅₀₁−S₅₀₂ and S_{comp1}=2×S_{ac501}+(_{Srand501}+S_{rand502}). Thus, this composite signal includes a signal component originating from the acoustic waves with an amplitude that is twice as large as that of the output signals from the acoustic wave detection elements 501 and 502. In the meantime, the composite signal has a reduced noise component originating from the motor or the like and has the random noise components added thereto.

The first composite signal according to the present exemplary embodiment is obtained by averaging signals from the acoustic wave detection elements in the first combining unit 533, as illustrated in FIG. 7. The composite signal input to an input terminal of each of the subtractors 532 has the random noise obtained by taking a root-mean-square of the random noise in the output signals from the acoustic wave detection elements.

A generalized case is described where the first composite signal is obtained by combining the output signals from n number of first acoustic wave detection elements 501 and the second composite signal is obtained by taking the output signal from a single second acoustic wave detection element 502 is described. In such a case, a second composite signal S_{compn} is expressed as S_{compn}=S₅₀₁ₙ/n+S₅₀₂ and as S_{compn}=2×S_{ac501}+((n+1)/n)^{0.5}×S_{rand501}. Thus, a smaller increase in the random noise in the second composite signal can be achieved with a larger number of elements involved for generating the first composite signal. More specifically, when two of the acoustic wave detection elements 501 are involved, the random noise component increases only by a factor of ((2+1)/2)^{0.5}=1.5^{0.5} (≈1.2). In other words, the resultant random noise component is 85% (=1.2/1.4) of that in the case of a 1 to 1 combining configuration. Meanwhile, the signal component based on the acoustic waves has doubled amplitude. Thus, the second composite signal has an S/N ratio improved by 17% (=1/0.85) over that in the case where a signal from a single acoustic wave detection element 501 and a signal from a single acoustic wave detection element 502 are combined.

As described above, an n to 1 combining configuration in which the second composite signal is obtained with n (≧2) number of acoustic wave detection elements 501 and a single acoustic wave detection element 502 can achieve a smaller increase in the random noise component, compared with that in the 1 to 1 combining configuration. The random noise component in the second composite signal can be generalized as ((n+1)/n)^{0.5} indicating that the random noise components increases by a factor of 1 with n→∞. Thus, the combining involves no increase in the random noise component with n→∞.

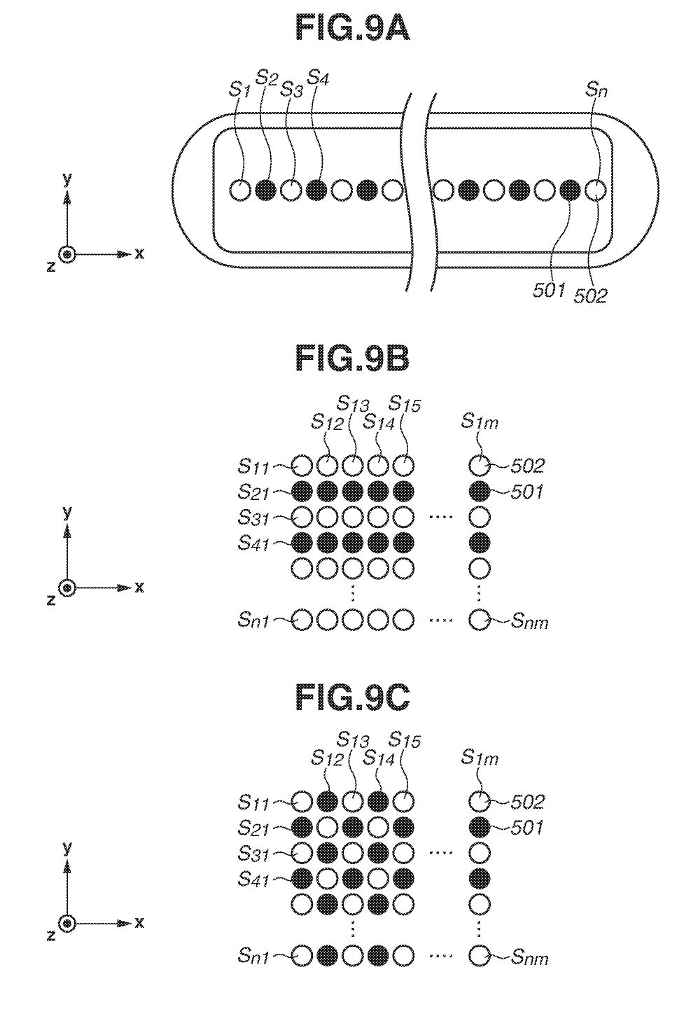

FIG. 9 illustrates alternative arrangements of the acoustic wave detection elements according to the present exemplary embodiments.

FIG. 9A illustrates an example of a one-dimensional arrangement of the first acoustic wave detection elements 501, represented by black circles, and the second acoustic wave detection elements 502, represented by white circles, being alternately arranged. In this arrangement example, the first composite signal is generated with a plurality of acoustic wave detection elements 501 (S2, S4) adjacent to an acoustic wave detection element 502 (S3). In this configuration, the first composite signal can be generated with a centroid of an acoustic wave reception surface of the plurality of acoustic wave detection elements 501 (S2, S4) substantially matching that of the acoustic wave detection element 502 (S3). Thus, a second composite signal Scomp2 with substantially no shifting of the centroid of the acoustic wave reception surface with respect to the acoustic wave detection element 502 (S3) can be obtained.

With the acoustic wave detection elements 501 and the acoustic wave detection elements 502 one-dimensionally arranged periodically and alternately as described above, a smaller increase in the random noise component can achieved. At the same time, the second composite signal can be obtained with no shifting of the centroid of the acoustic wave reception surface. The 2 to 1 combining configuration described above as an example should not be construed in a limiting sense. The number of acoustic wave detection elements involved in generating the first composite signal may be a number other than two, for example, four, as long the centroids of the acoustic wave reception surfaces match. In such a 4 to 1 combining configuration, the random noise component increases only by a factor of ((4+1)/4)0.5=1.250.5(≈1.1). Thus, this 4 to 1 combining configuration using the four acoustic wave detection elements 501 provided for a single acoustic wave detection element 502 can cut down the increased amount of the random noise component in the second composite signal by approximately 20% from, that in the 1 to 1 combining configuration. In other words, the random noise component that is 78% (=1.1/1.4) of that in the 1 to 1 combining configuration can be achieved.

Meanwhile, the signal component based on the acoustic waves has the amplitude doubled from that in the output from a single acoustic wave detection element, as in the case of the 1 to 1 combining configuration. Thus, the second composite signal with an S/N ratio improved by 27% (=1/0.78) over that in the 1 to 1 combining configuration can be achieved.

FIG. 9B illustrates an example of a two-dimensional arrangement of the plurality of acoustic wave detection elements 501 and the plurality of acoustic wave detection elements 502. In this arrangement, arrays each including the plurality of acoustic wave detection elements 501 arranged in an x axis direction and arrays each including the plurality of acoustic wave detection elements 502 similarly arranged in the x axis direction are alternately arranged in a y axis direction.

In this configuration, the first composite signal is obtained with two acoustic wave detection elements 501 (S21, S41) adjacent to a single acoustic wave detection element 502 (S31). In this configuration, the first composite signal can be generated with a centroid of the acoustic wave reception surface of the plurality of acoustic wave detection elements 501 (S21, S41) substantially matching that of the acoustic wave detection element 502 (S31). As a result, a second composite signal Scomp2 with substantially no shifting of the centroid of the acoustic wave reception surface can be obtained, with the centroid of the acoustic wave reception surfaces of the plurality of acoustic wave detection elements 501 (S21, S41) substantially matching that of the acoustic wave detection element 502 (S31).

Also in this example where the acoustic wave detection elements 501 and the acoustic wave detection elements 502 are alternately arranged, the second composite signal with an improved S/N ratio and no shifting of the centroid of the acoustic wave reception surface can be obtained.

In FIG. 9B, the acoustic wave detection elements 501 and the acoustic wave detection elements 502 are alternately arranged in the Y axis direction. Alternatively, the acoustic wave detection elements 501 and the acoustic wave detection elements 502 may be alternately arranged in the X axis direction.

FIG. 9C illustrates another exemplary two-dimensional arrangement.

In this arrangement example, the acoustic wave detection elements 501 and the acoustic wave detection elements 502 are arranged in a checkerboard pattern.

The first composite signal is obtained with four acoustic wave detection elements 501 (S12, S21, S23, and S31) adjacent to a single acoustic wave detection element 502 (S22). In this configuration, the combined signal from the plurality of acoustic wave detection elements 501 (S12, S21, S23, and S31) can be obtained with the centroid of the acoustic wave reception surface substantially matching that of the single acoustic wave detection element 502 (S22).

Thus, the first composite signal and a second composite signal Scomp4 from the single acoustic wave detection element 502 (S22) with substantially the same centroid of the acoustic wave reception surface can be obtained. Thus, the second composite signal Scomp4 can be obtained with substantially no shifting of the centroid of the acoustic wave reception surface.

Also in this example where the acoustic wave detection elements 501 and 502 are alternately arranged, the second composite signal with an improved S/N ratio and substantially no shifting of the centroid of the acoustic wave reception surface can be obtained.

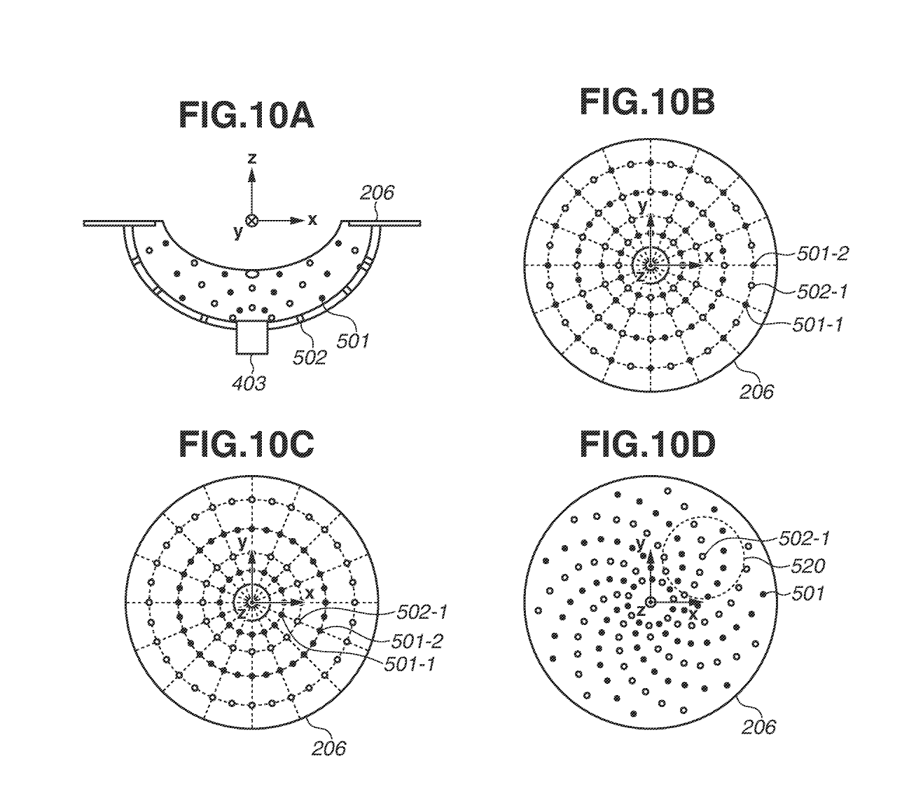

The present exemplary embodiment can be applied to a three-dimensional arrangement of the acoustic wave detection elements. FIG. 10 illustrates an alternative arrangement example of the acoustic wave detection elements according to present exemplary embodiment. In this example, the plurality of acoustic wave detection elements 501 and 502 are supported by a semispherical supporting member in such a manner that directional axes of the acoustic wave detection elements 501 and the acoustic wave detection elements 502 are concentrated at a point around the center of the semisphere. The shape of the supporting member is not limited to the semispherical shape and may be in any shape as long as the plurality of acoustic wave detection elements can be supported with the directional axes concentrated at a certain area.

FIG. 10A is a cross-sectional view of a configuration in which the plurality of acoustic wave detection elements 501 and the plurality of acoustic wave detection element 502 are supported by the semispherical supporting member, taken along an x-z plane.

FIG. 10B illustrates the supporting member as viewed in a z axis direction. The plurality of acoustic wave detection elements 501 and the plurality of acoustic wave detection elements 502 are alternately arranged on a plurality of concentric circles. In this configuration, the acoustic wave detection elements 501 and the acoustic wave detection elements 502 are arranged at an equal interval in a circumferential direction on each of the concentric circles, so that the first composite signal can obtained with two acoustic wave detection elements 501-1 and 501-2, adjacent to a single acoustic wave detection element 502-1 on the same circle, having the centroid of the acoustic wave reception surface substantially matching that of the single acoustic wave detection element 502-1. The first composite signal thus obtained is combined with the signal from the single acoustic wave detection element 502-1, whereby a second composite signal Scompn with an improved S/N ratio with substantially no shifting of the centroid of the acoustic wave reception surface can be obtained.

As described above, also with this semispherical arrangement of the plurality of acoustic wave detection elements 501 and the plurality of acoustic wave detection elements 502, the second composite signal Scompn with an improved S/N ratio and substantially no shifting of the centroid of the acoustic wave reception surface can be obtained.

FIG. 10C illustrates the plurality of acoustic wave detection elements 501 and the plurality of acoustic wave detection elements 502 arranged on the semispherical supporting member, as viewed in the z axis direction. In this arrangement example, the plurality of acoustic wave detection elements 501 and the plurality of acoustic wave detection elements 502 are alternately arranged in radial directions.

In this configuration, the acoustic wave detection elements 501 and the acoustic wave detection elements 502 are alternately arranged at an equal interval in the radial direction, so that the first composite signal can be generated with the two acoustic wave detection elements 501-1 and 501-2 adjacent to a single acoustic wave detection element 502-1, with the centroid of the acoustic wave reception surface substantially matching that of the single acoustic wave detection element 502-1.

The first composite signal thus obtained is combined with a signal from the single acoustic wave detection element 502-1 of interest, whereby the second composite signal Scompn with an improved S/N ratio and no shifting of the acoustic wave reception surface can be obtained. Thus, also in this configuration, the second composite signal Scompn with an improved S/N ratio and no shifting of the centroid of the acoustic wave reception surface can be obtained.

FIG. 10D illustrates an example of a semispherical arrangement of the plurality of acoustic wave detection elements 501 and the plurality of acoustic wave detection elements 502 as viewed in the z axis direction. In this example, the plurality of acoustic wave detection elements 501 and the plurality of acoustic wave detection elements 502 are arranged in a spiral form. Here, the acoustic wave detection element 502-1 is the acoustic wave detection element of interest.

The first composite signal is obtained from the plurality of acoustic wave detection elements 501 within a circular area 520 with the acoustic wave detection element 502-1 at the center. Thus, the centroid of the acoustic wave reception surface of the plurality of acoustic wave detection elements 501 can substantially match that of the acoustic wave detection element 502-1 of interest.

Because the centroid of the acoustic wave reception surface for the first composite signal substantially matches that of the acoustic wave detection element 502-1 of interest as described above, the second composite signal can be obtained with high positional accuracy. Thus, the second composite signal Scompn with an improved S/N ratio and no shifting of the centroid of the acoustic wave reception surface can be obtained, also when the plurality of acoustic wave detection elements 501 and the plurality of acoustic wave detection elements 502 are arranged in the spiral form.

A similar effect can be obtained when this method of obtaining the first composite signal from the plurality of acoustic wave detection elements 501 within the area 520 is applied to other one-dimensional or two-dimensional arrangements of the acoustic wave detection element.

The same processing as those for the arrangements illustrated in FIG. 10B and FIG. 10C may be executed by using the acoustic wave detection element 502 and the plurality of acoustic wave detection elements 501 adjacent thereto, with the acoustic wave detection elements 502 and the acoustic wave detection elements 501 alternately arranged on a single spiral. Also with this configuration, the second composite signal Scompn with a smaller increase in the random noise component and no shifting of the centroid of the acoustic wave reception surface can be obtained. Thus, the second composite signal Scompn with the S/N ratio improved over that in the 1 to 1 combining configuration and no shifting of the centroid of the acoustic wave reception surface can be obtained, with the plurality of acoustic wave detection elements 501 and the plurality of acoustic wave detection elements 502 alternately arranged in any one of spiral, circumferential, and radial directions.

With the present exemplary embodiment described above, image processing can be executed based on the second composite signal Scompn with an improved S/N ratio and no shifting of the centroid of the acoustic wave reception surface obtained by combining a signal from the single acoustic wave detection element 502 with the first composite signal obtained by combining the output signals from the plurality of acoustic wave detection elements 501. Thus, an excellent reconstructed image can be obtained.

In the example described above, the combining unit 533 is provided for combining the output signals from the first acoustic wave detection elements 501 only. Alternatively, the combining unit 533 may be similarly provided to the second acoustic wave detection elements 502 to further improve the S/N ratio of the electric signal. Thus, a configuration in which composite signals with opposite polarities are respectively input to the input terminals of the subtractor 532 may be employed.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Applications No. 2016-137645, filed Jul. 12, 2016, and No. 2017-118014, filed Jun. 15, 2017, which are hereby incorporated by reference herein in their entirety.

What is claimed is:

1. A subject information acquisition apparatus, comprising:
at least one first detection element and at least one second detection element, wherein the first detection element is configured to output signals with polarities opposite to the second detection element, upon receiving acoustic waves propagated from a subject; and
a signal processing unit configured to acquire information on the subject by using a differential signal obtained based on a difference between signals output from the first and the second detection elements.

2. The subject information acquisition apparatus according to claim 1, further comprising:
a plurality of the second detection elements, wherein the differential signal is based on a difference between the signal output from the first detection element and a composite signal obtained by combining a plurality of signals output from the plurality of the second detection elements.

3. The subject information acquisition apparatus according to claim 1, wherein the first and the second detection elements each include:
a detection unit configured to detect the acoustic waves and output a detection signal; and
an amplifying unit configured to amplify the detection signal.

4. The subject information acquisition apparatus according to claim 3,
wherein the detection unit is a capacitive pressure sensitive element having one terminal connected to the amplifying unit and another terminal connected to a power source, and
wherein the first and the second detection elements are opposite to each other in how the capacitive pressure sensitive element is connected to the amplifying unit and the power source.

5. The subject information acquisition apparatus according to claim 3,
wherein the detection unit is a piezoelectric element having one terminal connected to the amplifying unit and another terminal connected to the power source, and
wherein the first and the second detection elements are opposite to each other in how the piezoelectric element is connected to the amplifying unit and the power source.

6. The subject information acquisition apparatus according to claim 1, further comprising:
a supporting member configured to support the first and the second detection elements; and
a driving unit configured to move the supporting member with respect to the subject.

7. The subject information acquisition apparatus according to claim 6, further comprising:
a plurality of the first and the second detection elements, wherein the supporting member is configured to support the plurality of the first and the second detection elements in such a manner that directional axes of the plurality of the first and the second detection elements are concentrated.

8. The subject information acquisition apparatus according to claim 1,
wherein the first and the second detection elements are formed as a single module, and
wherein the subject information acquisition apparatus includes a plurality of the modules.

9. The subject information acquisition apparatus according to claim 1, further comprising a light emitting unit configured to irradiate the subject with light,
wherein the acoustic waves are generated as a result of irradiating the subject with the light.

10. The subject information acquisition apparatus according to claim 1, further comprising a determination unit configured to determine whether the difference between the signal output from the first detection element and the signal output from the second detection element exceeds a first threshold.

11. The subject information acquisition apparatus according to claim 10, wherein the determination unit is further configured to determine whether the difference between the signal output from the first detection element and the signal output from the second detection element exceeds a second threshold larger than the first threshold.

12. The subject information acquisition apparatus according to claim 10, further comprising a plurality of the first detection elements and a plurality of the second detection elements,
wherein the signal processing unit is configured to acquire, when the determination unit determines that a difference between signals respectively output from one of the first detection elements and one of the second detection elements exceeds the first threshold, information on the subject by using signals output from the first and the second detection elements other than the one of the first detection elements and the one of the second detection elements outputting the signals the difference between which has been determined to exceed the first threshold.

13. A method for acquiring subject information, the method comprising:
outputting, by a pair of detection elements upon receiving acoustic waves, signals with polarities opposite to each other, and
acquiring information on a subject by using a differential signal obtained based on a difference between the signals with polarities opposite to each other.

14. The method for acquiring subject information according to claim 13, further comprising:
combining a signal output from a detection element other than the pair of detection elements with one of the signals output from the pair of detection elements with a same polarity, to obtain a composite signal; and
acquiring the differential signal obtained based on a difference between the composite signal and the signal output from another one of the pair of detection elements.

15. The method for acquiring subject information according to claim 13, the method further comprising determining whether the differential signal exceeds a first threshold.

16. The method for acquiring subject information according to claim 15, the method further comprising determining whether the differential signal exceeds a second threshold larger than the first threshold.

17. The method for acquiring subject information according to claim 15, the method further comprising acquiring, when the differential signal obtained from one of a plurality of the pairs of the detection elements is determined to exceed the first threshold, information on the subject by using the differential signal output from another one of the pairs of the detection elements different from the one of the pairs of the detection elements outputting the differential signal determined to exceed the first threshold.

18. A subject information acquisition apparatus comprising:
a plurality of first detection elements and a second detection element, wherein the plurality of first detection elements is configured to output signals with polarities opposite to the second detection element, upon receiving acoustic waves propagated from a subject;
a first combining unit configured to combine the signals output from the plurality of first detection elements to output a first composite signal; and
a second combining unit configured to combine the first composite signal with a signal output from the second detection element to output a second composite signal.
